(19) United States
(12) Patent Application Publication
(10) Pub. No.: US 2021/0290859 A1
(43) Pub. Date: Sep. 23, 2021

(54) DENTAL TOOL

(71) Applicant: THE RESEARCH FOUNDATION FOR THE STATE UNIVERSITY OF NEW YORK, Albany, NY (US)

(72) Inventor: Allan KUCINE, Albany, NY (US)

(73) Assignee: THE RESEARCH FOUNDATION FOR THE STATE UNIVERSITY OF NEW YORK, Albany, NY (US)

(21) Appl. No.: 17/258,918

(22) PCT Filed: Jul. 10, 2019
(86) PCT No.: PCT/US2019/041168

§ 371 Date: Jan. 8, 2021

Related U.S. Application Data
(60) Provisional application No. 62/696,427, filed on Jul. 11, 2018.

(51) Int. Cl. A61M 5/42; A61C 1/08; A61M 19/00

(52) U.S. Cl. CPC A61M 5/427; A61M 2210/0625; A61M 19/00; A61C 1/082

(57) ABSTRACT

The present disclosure is directed to a dental tool and methods for using the same. The dental tool includes a handle; a tip operably attached to a first end of the handle; and an alignment element that is substantially parallel with the handle.

### CROSS REFERENCE TO RELATED APPLICATIONS

This application claims benefit of U.S. Provisional Application No. 62/696,427, filed Jul. 11, 2018, the entire contents of which is incorporated hereby by reference.

### BACKGROUND OF THE DISCLOSURE

The greatest risk of exposure of health care providers to blood borne pathogens (e.g. HBV, HCV, HIV) occurs during the handling/use of suture needles and injection needles. Despite this risk, the administration of local anesthetics in dentistry often entails the use of the non-dominant hand of the dentist as an intraoral retractor.

Typically, it is a requirement for the dentist to palpate the region to locate anatomic landmarks to more accurately place the syringe and needle when administering the local anesthetic. However, the dentist will often then keep finger(s) of the non dominant hand in the oral cavity to maintain tension on the oral mucosa, and orientation with known landmarks.

In particular, the inferior alveolar nerve block, which is commonly administered in the practice of dentistry, is dependent on utilizing specific anatomic landmarks, such as the coronoid notch (anterior border of the ascending ramus) of the mandible. The dentist needs to determine the deepest part of this curvilinear notch, as the location of the inferior alveolar nerve is typically on this plane, but approximately 25 mm deep within the tissue (thus, close to a nerve). The dentist typically utilizes their thumb or forefinger on their non-dominant hand for this purpose.

Furthermore, the complexity and normal variation of the anatomy, combined with the depth of the injection result in a significant failure rate. Failure rates of up to 20% have been reported.

Hence, there is a need for a device that can enhance usability and accuracy for the operator, as well as keep the operators fingers away from the needle and injection site to enhance safety for the operator.

Embodiments of the present disclosure provide devices and methods that address the above needs.

### SUMMARY OF THE DISCLOSURE

The present disclosure is directed to dental tool. The dental tool comprises a handle; a tip operably attached to a first end of the handle; and an alignment element that is substantially parallel with the handle.

In another embodiment, the disclosure is directed to a method of delivering an injection to a patient. The method comprises applying pressure to a coronoid notch of a mandible with a surface of a dental tool to stretch a surface of the tissue laterally; substantially aligning a needle of a syringe along an alignment element of the dental tool, so that the needle enters tissue while being in substantial alignment with the alignment element; and injecting a liquid from within the syringe into the tissue while maintaining the needle in substantial alignment with the alignment element.

### BRIEF DESCRIPTION OF THE DRAWINGS

The present disclosure will be better understood by reference to the following drawings, which are provided as illustrative of certain embodiments of the subject application, and not meant to limit the scope of the present disclosure.

FIG. 1 is a graphical illustration of a mandible of a human.

FIG. 2 is a graphical illustration of an embodiment of a dental tool.

FIG. 3 is a graphical illustration of an embodiment of a dental tool.

FIG. 4 is a graphical illustration of an embodiment of a dental tool and syringe within a patient's mouth.

FIG. 5 is a graphical illustration of a top view of another embodiment of a dental tool.

FIG. 6 is a graphical illustration of a cross-sectional view of the other embodiment of the dental tool.

FIG. 7 is a graphical illustration of a perspective view of the other embodiment of the dental tool.

### DETAILED DESCRIPTION OF THE DISCLOSURE

In the discussion and claims herein, the term “about” indicates that the value listed may be somewhat altered, as long as the alteration does not result in nonconformance of the process or device. For example, for some elements the term “about” can refer to a variation of ±0.1%, for other elements, the term “about” can refer to a variation of ±1% or ±10%, or any point therein.

As used herein, the term “substantially”, or “substantial”, is equally applicable when used in a negative connotation to refer to the complete or near complete lack of an action, characteristic, property, state, structure, item, or result. For example, a surface that is “substantially” flat would either completely flat, or so nearly flat that the effect would be the same as if it were completely flat.

As used herein terms such as “a”, “an” and “the” are not intended to refer to only a singular entity, but include the general class of which a specific example may be used for illustration.

As used herein, terms defined in the singular are intended to include those terms defined in the plural and vice versa.

References in the specification to “one embodiment”, “certain embodiments”, some embodiments” or “an embodiment”, indicate that the embodiment(s) described may include a particular feature or characteristic, but every embodiment may not necessarily include the particular feature, structure, or characteristic. Moreover, such phrases are not necessarily referring to the same embodiment. Further, when a particular feature, structure, or characteristic is described in connection with an embodiment, it is submitted that it is within the knowledge of one skilled in the art to affect such feature, structure, or characteristic in connection with other embodiments whether or not explicitly described. For purposes of the description hereinafter, the terms “upper”, “lower”, “right”, “left”, “vertical”, “horizontal”, “top”, “bottom”, and derivatives thereof shall relate to the invention, as it is oriented in the drawing figures. The terms “overlying”, “atop”, “positioned on” or “positioned atop” means that a first element, is present on a second element, wherein intervening elements interface between the first element and the second element. The term “direct contact” or “attached to” means that a first element, and a second element, are connected without any intermediary element at the interface of the two elements.

Reference herein to any numerical range expressly includes each numerical value (including fractional numbers and whole numbers) encompassed by that range. To illustrate, reference herein to a range of “at least 50” or “at least about 50” includes whole numbers of 50, 51, 52, 53, 54, 55, 56, 57, 58, 59, 60, etc., and fractional numbers 50.1, 50.2 50.3, 50.4, 50.5, 50.6, 50.7, 50.8, 50.9, etc. In a further illustration, reference herein to a range of “less than 50” or “less than about 50” includes whole numbers 49, 48, 47, 46, 45, 44, 43, 42, 41, 40, etc., and fractional numbers 49.9, 49.8, 49.7, 49.6, 49.5, 49.4, 49.3, 49.2, 49.1, 49.0, etc.

The disclosure is directed to a dental tool that can be used to assist various dental procedures, such as oral injections. The dental tool can be used in conjunction of any liquid material orally, such as, for example, a local anesthetic.

This dental tool is specifically and anatomically designed to locate the deepest part of the coronoid notch, maintain tissue tension, and retract the cheek during an injection, such as an inferior alveolar nerve block injection, thereby allowing the operator to perform the injection safely and accurately.

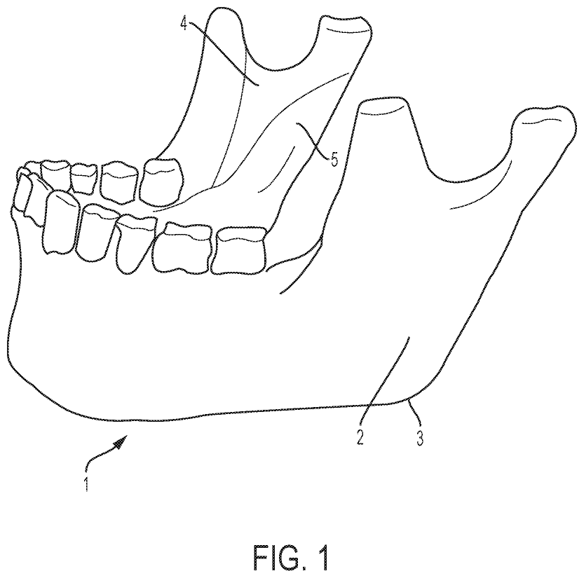

As can be seen in FIG. 1, a human mandible 1 is illustrated, which is also known as a lower jaw. Mandible 1 includes ramus 2, which is substantially quadrilateral in shape and has two surfaces, a lateral substantially flat surface and a medial surface 4. The medial surface 4 presents about its center the mandibular foramen 5, for the entrance of inferior alveolar vessels and nerves. The medial pterygoid (or internal pterygoid muscle), is a relatively thick, substantially quadrilateral muscle of mastication. Its fibers pass downward, lateral, and posterior, and are inserted, by a strong tendinous lamina, into the lower and back part of the medial surface 4 of the ramus 2 and an angle of the mandible 3, as high as the mandibular foramen 5.

Mandibular foramen 5 on the internal surface of ramus 2 is an efficient location for administering anesthesia to a patient. Generally, the anesthesia is administered near the mandible foramen 5 to cause blockade of the nerve and the nearby lingual nerve for subsequent dental procedures.

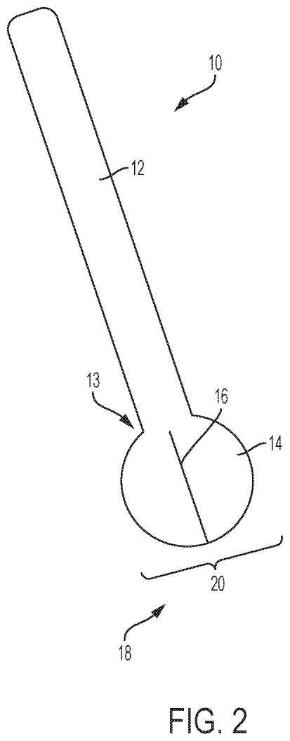

As can be seen in FIG. 2, a top view of a dental tool 10 configured for placement on the coronoid notch on the anterior surface of the ramus is shown. The dental tool 10 includes a handle 12 and a tip 14. In some embodiments, the handle 12 is operably connected to the tip 14 at a distal end 13 of the handle 12, in other embodiments, the handle 12 and tip 14 are a single piece of material. The dental tool 10 can be formed of any material, such as a resin, a plastic, a glass, a metal, a carbon based material, and combinations thereof.

The surface of the tip 14 shown in FIG. 2 can be substantially flat, can be substantially concave or can be substantially convex. In the embodiments where the surface of the tip 14 is concave, this concavity can form a pocket that a volume of a patient's buccal mucosa (cheek) can enter, which can add to the comfort of the patient.

An alignment element 16 is seen as extending a length of the tip 14. Alignment element 16 is a visual indicator, which can be raised from a surface of the tip 14, be coplanar with the tip 14 or be embedded within, but visible externally to, the tip 14. The alignment element 16 can be formed as a single piece of material from the tip 14, or, the alignment element 16 can be operably attached to the tip 14, or in other embodiments, the alignment element 16 can be an ink or paint applied to a surface of the tip 14, or embedded within, but visible externally to, the tip 14.

A distal end 18 of tip 14 is the portion of the dental tool 10 configured for placement on the coronoid notch on the anterior surface of the ramus. Specifically, a contact portion 20 of the tip, shown approximately as the area enclosed by bracket 20, is configured to contact and be placed on the coronoid notch on the anterior surface of ramus.

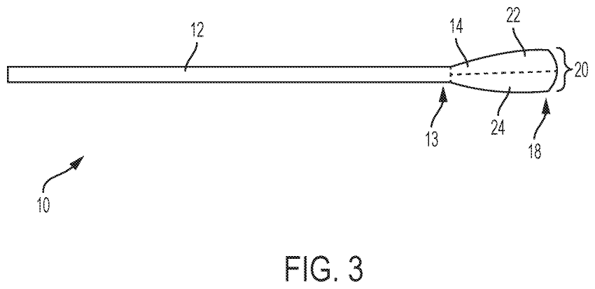

More detail of the contact portion 20 can be seen in FIG. 3, which is a side view of the dental tool 10. Contact portion 20 is shaped to match or substantially match the shape of a human's coronoid notch on the anterior surface of ramus.

As can be seen in FIG. 3, the thickness of the tip 14 increases from the distal end 13 of the handle 12 to the distal end 18 of tip 14.

The dotted lines in FIG. 3 are added for illustrative purposes only, and in FIG. 3, the dotted lines separate an upper section 22 of the tip 14 from a lower section 24 of the tip 14 and also separates the upper section 22 of the tip 14 and the lower section 24 of the tip 14 from the handle 12. The handle 12 is shown as being substantially straight in FIGS. 2 and 3, but in other embodiments, handle 12 can be curved and/or angled in any axis to any desired degree. Also, the handle 12 can be formed so as to be rigid, substantially rigid, somewhat flexible or flexible, as desired.

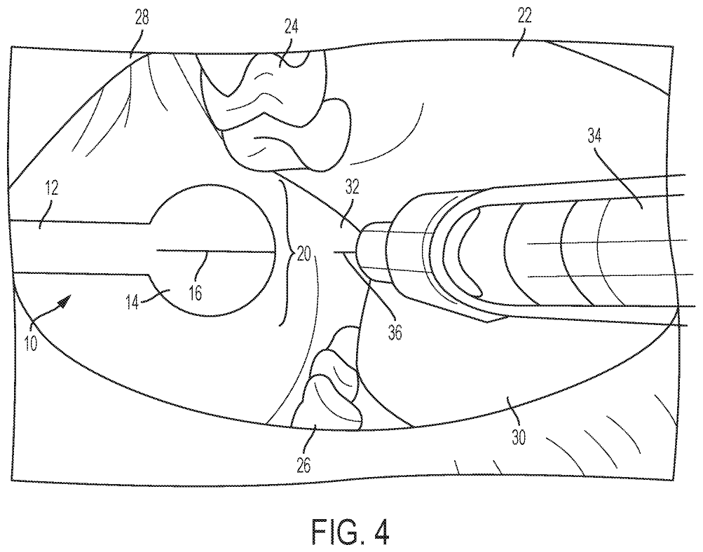

A method of using the dental tool 10 is described below with reference to FIG. 4.

Initially, a user grasps the handle 12 of the dental tool 10 places the dental tool 10 within a mouth 22 of a patient. As can be seen in FIG. 4, the dental tool 10 is moved between the patient's upper teeth 24 and the patient's lower teeth 26, on the patient's inner surface of their buccal mucosa (cheek) 28, away from the patient's tongue 30.

The contact portion 20 is placed in contact with the buccal mucosa 28 at a posterior point and lateral to a pterygomandibular raphe 32. A pressure is then applied, by the contact portion 20, to a coronoid notch of the patient's mandible (not shown, but shown in FIG. 1). The user can then move the contact portion 20 anteriorly and laterally away from the tongue 30, which is the position shown in FIG. 4. During any portion of this method, the handle 12 of the dental tool may rest or come in contact with a commissure (not shown) of the patient's mouth.

Another user, or the same user with their other hand, grasps a syringe 34, in which a liquid is contained, such as a local anastethetic. The user then aligns a needle 36 of the syringe 34 so that the needle 36 is offset and substantially parallel with the alignment element 16. Depending on the desired outcome, the offset distance between the placement of the needle 36 and the contact portion 20 can be modified, and in some embodiments can be about 5 mm, about 10 mm, about 15 mm, about 20 mm, about 25 mm or greater.

The user then causes the needle 36 to enter tissue, while maintaining substantial alignment with the alignment element 16. Following entrance into the tissue and advancement to the desired depth or location, the user can inject the liquid from the syringe while maintaining the needle 36 in substantial alignment with the alignment element.

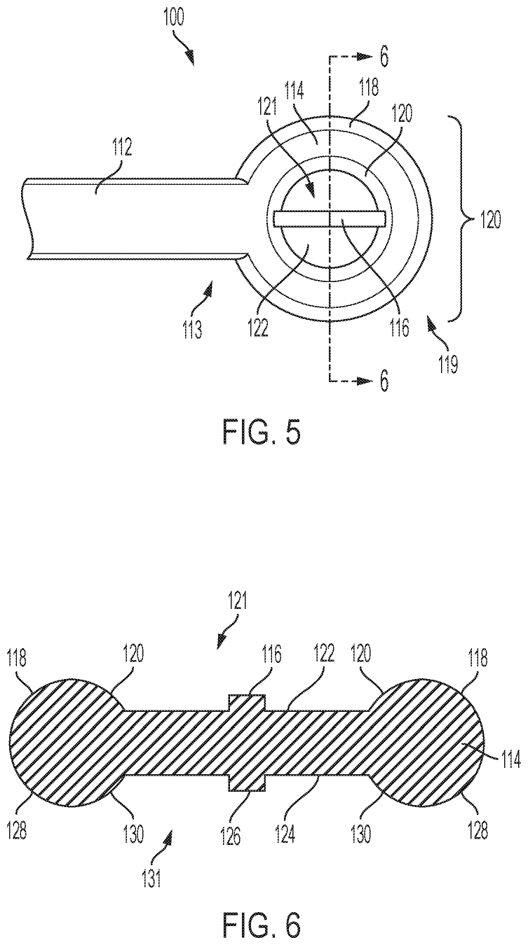

Another embodiment of a dental tool is shown in FIG. 5. FIG. 5 is a top view of a tip 114 of a dental tool 100. The tip 114 is connected to a handle 112, at a distal end 113 of the handle 112. A distal end 119 of tip 114 is the portion of the dental tool 100 configured for placement on the coronoid notch on the anterior surface of the ramus. Specifically, a contact portion 120 of the tip, shown approximately as the area enclosed by bracket 120, is configured to contact and be placed on the coronoid notch on the anterior surface of ramus.

In this embodiment, a concave portion 121 is formed on the surface of the tip 114 The concave portion 121 includes a substantially flat portion 122 and includes an alignment element 116 extending a length of the tip 114, along the substantially flat portion 122. The concave portion 121 includes an inner curved portion 120 and an outer curved portion 118, which are shown in FIG. 5 but better seen in FIG. 6.

The alignment element 116 protrudes vertically above the substantially flat portion 133 a distance towards the intersection of the inner curved portion 120 and the outer curved portion 118, but does not extend above the intersection of the inner curved portion 120 and the outer curved portion 118, which is shown in the cross-section view of FIG. 6.

FIG. 6 is a cross sectional view along the line illustrated in FIG. 5. As can be seen, the tip 114 can include the concave portion 121, and a lower concave portion 131. However, in other embodiments, the lower concave portion 131 is not present and the lower surface of the tip 114 is substantially flat across its surface area.

In FIG. 6, the lower concave portion 131 includes a lower inner curved portion 130 and a lower outer curved portion 128. The lower concave portion 131 includes a lower substantially flat portion 124 (similar in shape and appearance to substantially flat portion 122) and includes a lower alignment element 126 (similar in shape and appearance to alignment element 116) extending a length of the tip 114, along the lower substantially flat portion 124.

As can be seen in FIG. 6, in this embodiment the tip 114 is substantially the same thickness from the distal end (113 of FIG. 5) to the distal end (119 of FIG. 5), with that thickness being about 5 mm. However, in other embodiments, the thickness can be in the range of about 4 mm to about 6 mm, about 3 mm to about 7 mm, about 2 mm to about 8 mm, less than about 2 mm, or greater than about 8 mm.

Also as can be seen in FIG. 6, the alignment element 116 is substantially the same width as the lower alignment element 126. However, in other embodiments, one of the alignment element 116 and the lower alignment element 126 can be wider than the other.

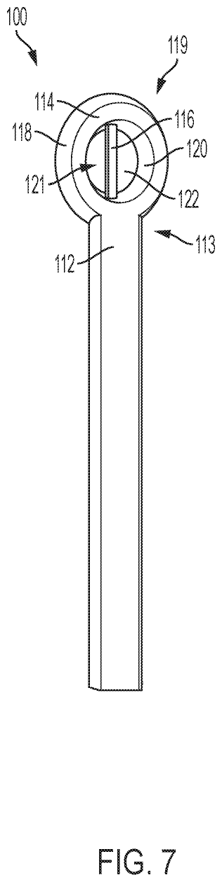

FIG. 7 provides yet another view, in this figure a perspective view, of the dental tool 100.

The described embodiments and examples of the present disclosure are intended to be illustrative rather than restrictive, and are not intended to represent every embodiment or example of the present disclosure. While the fundamental novel features of the disclosure as applied to various specific embodiments thereof have been shown, described and pointed out, it will also be understood that various omissions, substitutions and changes in the form and details of the devices illustrated and in their operation, may be made by those skilled in the art without departing from the spirit of the disclosure. For example, it is expressly intended that all combinations of those elements and/or method steps which perform substantially the same function in substantially the same way to achieve the same results are within the scope of the disclosure. Moreover, it should be recognized that structures and/or elements and/or method steps shown and/or described in connection with any disclosed form or embodiment of the disclosure may be incorporated in any other disclosed or described or suggested form or embodiment as a general matter of design choice. Further, various modifications and variations can be made without departing from the spirit or scope of the disclosure as set forth in the following claims both literally and in equivalents recognized in law.

1. A dental tool comprising:
a handle;
a tip operably attached to a first end of the handle; and
an alignment element that is substantially parallel with the handle.

2. The dental tool of claim 1, wherein the handle is rigid.

3. The dental tool of claim 1, wherein the handle is substantially rigid.

4. The dental tool of claim 1, wherein the tip comprises a contact portion.

5. The dental tool of claim 4, wherein the contact portion is dimensioned to match or substantially match the shape of a human's coronoid notch on the anterior surface of the ramus.

6. The dental tool of claim 1, wherein a thickness of the tip increases from a distal end of the handle to a distal end of tip.

7. The dental tool of claim 1, wherein a surface of the tip comprises a concave portion.

8. The dental tool of claim 7, wherein the concave portion comprises a substantially flat portion. The dental tool of claim 8, wherein the alignment element extends along the substantially flat portion.

10. The dental tool of claim 7, wherein the alignment element protrudes a distance vertically above the substantially flat portion.

11. The dental tool of claim 7, wherein a surface of the tip comprises a lower concave portion.

12. The dental tool of claim 11, wherein the lower concave portion comprises a lower substantially flat portion.

13. The dental tool of claim 12, wherein a lower alignment element extends along the lower substantially flat portion.

14. The dental tool of claim 13, wherein the lower alignment element protrudes a distance vertically above the substantially flat portion.

15. The dental tool of claim 13, wherein a width of the alignment element is less than a width of the lower alignment element.

16. A method of delivering an injection to a patient, the method comprising:
applying pressure to a coronoid notch of a mandible with a surface of a dental tool to stretch a surface of the tissue laterally;
substantially aligning a needle of a syringe along an alignment element of the dental tool, so that the needle enters tissue while being in substantial alignment with the alignment element; and
injecting a liquid from within the syringe into the tissue while maintaining the needle in substantial alignment with the alignment element.

17. The method of claim 16, further comprising the step of contacting a commissure of the patient's mouth with a handle of the dental tool.
